(19) United States
(12) Patent Application Publication
(10) Pub. No.: US 2013/0156681 A1
(43) Pub. Date: Jun. 20, 2013

(54) Processes and Systems for Oxidizing Aqueous Metal Bromide Salts

(71) Applicant: Marathon GTF Technology, Ltd., Houston, TX (US)

(72) Inventors: Sabah A. Kurukchi, Houston, TX (US); Anand Moodley, Mpumalanga (ZA); Yijun Liu, Houston, TX (US)

(73) Assignee: MARATHON GTF TECHNOLOGY, LTD., Houston, TX (US)

(21) Appl. No.: 13/705,111

(22) Filed: Dec. 4, 2012

Related U.S. Application Data
(60) Provisional application No. 61/577,386, filed on Dec. 19, 2011.

(51) Int. Cl. C01B 7/09; C07C 1/30

(52) U.S. Cl. CPC C01B 7/096; C07C 1/30
USPC 423/500; 585/310; 422/162

(57) ABSTRACT

Processes and systems that include use of a packed wet oxidation reactor for oxidizing aqueous metal bromide salts in a bromine-based process for converting lower molecular weight alkanes to higher molecular weight hydrocarbons. A stream comprising a dissolved metal bromide salt may be oxidized in a wet oxidation reactor comprising a packed section to produce at least a partially oxidized liquid stream comprising oxidized products of the metal bromide salt and a gaseous bromine stream comprising elemental bromine.

### BACKGROUND OF THE INVENTION

The present invention relates generally to processes and systems for oxidizing aqueous metal bromide salts and, in one or more embodiments, to processes and systems that include use of a packed wet oxidation reactor for oxidizing aqueous metal bromide salts in a bromine-based process for converting lower molecular weight alkanes to higher molecular weight hydrocarbons.

Natural gas, a fossil fuel, is primarily composed of methane and other light alkanes and has been discovered in large quantities throughout the world. When compared to other fossil fuels, natural gas is generally a cleaner energy source. For example, crude oil typically contains impurities, such as heavy metals, which are generally not found in natural gas. By way of further example, burning natural gas produces far less carbon dioxide than burning coal. However, challenges are associated with the use of natural gas in place of other fossil fuels. Many locations in which natural gas has been discovered are far away from populated regions and, thus, do not have significant pipeline structure and/or market demand for natural gas. Due to the low density of natural gas, the transportation thereof in gaseous form to more populated regions can be expensive. Accordingly, practical and economic limitations exist to the distance over which natural gas may be transported in its gaseous form.

Cryogenic liquefaction of natural gas to form liquefied natural gas (often referred to as “LNG”) is often used to more economically transport natural gas over large distances. However, this LNG process is generally expensive, and there are limited regasification facilities in only a few countries for handling the LNG. Converting natural gas to higher molecular weight hydrocarbons which, due to their higher density and value, are able to be more economically transported as a liquid can significantly expand the market for natural gas, particularly stranded natural gas produced far from populated regions. While a number of processes for the conversion of natural gas to higher molecular weight hydrocarbons have been developed, these processes have not gained widespread industry acceptance due to their limited commercial viability. Typically, these processes suffer from undesirable energy and/or carbon efficiencies that have limited their use.

One technique for converting natural gas to higher molecular weight hydrocarbons is a bromine-based process. In general, the bromine-based process includes two gas-phase reactions: 1) bromination of natural gas using elemental bromine; and 2) reaction of the brominated alkanes via dehydrobromination into higher molecular weight hydrocarbons. Both of these gas-phase reactions generate hydrogen bromide (“HBr”) as a co-product. For practical and economic reasons, the HBr is generally separated from the higher molecular weight hydrocarbons and then oxidized to elemental bromine for reuse in the bromination reaction. Because of the large quantity and corrosiveness of HBr, an efficient and effective approach for its recovery can have significant impact on the overall feasibility and economics of the bromine-based process for converting natural gas to higher molecular weight hydrocarbons.

Some processes have been proposed for recovery of HBr from gaseous hydrocarbons followed by conversion of HBr to elemental bromine via a method that includes HBr neutralization with subsequent wet oxidation. In such processes, the HBr contained in the gaseous mixture is neutralized by contacting it with an aqueous solution comprising a metal hydroxide, a metal oxy-bromide species, or a combination thereof such that the HBr is neutralized to form a metal bromide salt in the aqueous solution. The aqueous solution containing the metal bromide salt then proceeds to a wet oxidation reactor wherein it is oxidized to yield elemental bromine and an aqueous solution of metal hydroxide, metal oxide, metal oxy-bromide, or mixtures of these species that can be reused for neutralization.

Wet oxidation has been used for the treatment of aqueous streams for over sixty years. In general, wet oxidation is the oxidation of one or more components in water using oxygen as the oxidizing agent. When air is used as the source of the oxygen, the oxidation is commonly referred to as wet air oxidation. The wet oxidation process typically involves the addition of air or oxygen to an aqueous stream at elevated temperatures and pressures, with the resultant “combustion” of oxidizable material directly within the aqueous phase. The largest application of wet oxidation is for the conditioning of wastewater, such as municipal sludge. Additional applications of wet oxidation include the treatment of pulp and paper mill effluents, spent caustic treatments to oxidize sodium sulfide to sulfate, and treatment of chemical plant effluents.

Conventional wet oxidation systems typically employ bubble column reactors, where the oxidizing agent is bubbled through a vertical column that is full of the hot and pressurized wastewater. Bubble column reactors are suitable for conventional wet oxidation systems to provide the necessary gas-liquid mass transfer with gas superficial velocities of about 0.05 meters per second. The wastewater enters the bottom of the column and oxidized wastewater exits the top. While wet oxidation systems have been used for treatment of wastewater with dilute organic or inorganic contaminants, these oxidation reactions typically require low air-to-liquid flow rates and residence times in the order of hours or even days. To accommodate the residence times, wet oxidation reactors for oxidative degradation are normally large. Thus, in addition to elevated pressure and temperature, homogeneous or heterogeneous catalysts are often used to accelerate the oxidation rate.

In contrast to these conventional wet oxidation systems, the high concentrations of metal bromide salt in the aqueous streams in the previously mentioned technique for HBr recovery require both high air-to-liquid flow rates and high gas-liquid mass transfer area. Accordingly, application of the conventional wet oxidation systems to HBr recovery is not deemed economically viable as impractical reactor diameters for industrial applications would be required at the gas superficial velocities needed for bubble column reactors.

Thus, a need exists for economically viable processes and systems for oxidizing aqueous metal bromide salts in a bromine-based process for converting lower molecular weight alkanes to higher molecular weight hydrocarbons.

### BRIEF SUMMARY OF THE INVENTION

To achieve the foregoing and other objects, and in accordance with the purposes of the present invention, as embodied and broadly described herein, one embodiment of the present invention is a process that comprises oxidizing a stream comprising a dissolved metal bromide salt in a wet oxidation reactor. The wet oxidation reactor may comprise a packed section to produce at least a partially oxidized liquid stream partially oxidized liquid stream comprising oxidized products of the metal bromide salt and a gaseous bromine stream comprising elemental bromine.

Another embodiment of the present invention is a process comprising reacting lower molecular weight alkanes and bromine to produce at least alkyl bromides and hydrogen bromide, wherein the lower molecular weight alkanes comprise methane. The process may further comprise reacting the alkyl bromides over a catalyst to produce at least higher molecular weight hydrocarbons and additional hydrogen bromide, wherein the higher molecular weight hydrocarbons comprise hydrocarbons having four or more carbon atoms. The process may further comprise neutralizing the hydrogen bromide and the additional hydrogen bromide in an aqueous stream of ferric hydroxide and ferrous bromide to produce at least a ferrous/ferric stream. The ferrous/ferric stream may comprise a dissolved ferrous bromide and a dissolved ferric bromide in a concentration of about 40 weight percent to about 60 weight percent. The process may further comprise oxidizing the ferrous/ferric stream in a wet oxidation reactor comprising a packed section to produce at least a partially oxidized liquid stream comprising ferric bromide. The wet oxidation reactor may operate at a temperature of about 140° C. to about 190° C. and a pressure of about 3 bars to about 20 bars.

Another embodiment of the present invention is a system comprising a wet oxidation reactor having an inlet in an upper end thereof for an aqueous metal bromide salt, an outlet in the lower end thereof for an oxidant stream, and one or more packed sections. The wet oxidation reactor may be configured to countercurrently contact the aqueous metal bromide salt with the oxidant stream over the one or more packed sections so as to oxidize the aqueous metal bromide salt and produce at least a partially oxidized liquid stream comprising oxidized products of the aqueous metal bromide salt and a gaseous bromine stream comprising elemental bromine.

### BRIEF DESCRIPTION OF THE DRAWINGS

The accompanying drawings, which are incorporated in and form a part of the specification, illustrate the embodiments of the present invention and, together with the description, serve to explain the principles of the invention.

In the drawings:

FIG. 1 is a schematic diagram of one embodiment of a process of the present invention; and

FIG. 2 is a schematic diagram of another embodiment of a process of the present invention.

### DETAILED DESCRIPTION OF THE PREFERRED EMBODIMENTS

The term “aqueous metal bromide salt” as used herein refers to an aqueous liquid that comprises a metal bromide salt dissolved therein. The metal bromide salt dissolved in the aqueous liquid may be any of a variety of different oxidizable metal salts. In some embodiments, the metal of the bromide salt may include transition metals, such as Fe(II), Cu(I), or mixtures thereof, as these transitional metals are less expensive and readily oxidizable at lower temperatures (e.g., about 120° C. to about 180° C.). It should be understood that Co, Ni, Mn, V, Cr, or other transition metals that form oxidizable bromide salts may also be used in alternative embodiments.

Suitable sources that may generate the aqueous metal bromide salt in various embodiments of the present invention include, but are not limited to, HBr. For example, the aqueous metal bromide salt may be generated by contacting a gaseous stream comprising hydrocarbons and HBr with an aqueous solution comprising a metal hydroxide, a metal oxide, or mixtures of these species to neutralize the HBr. As will be discussed in more detail below, the aqueous metal bromide salt may then be oxidized to yield elemental bromine. The elemental bromine generated by oxidation of the aqueous metal bromide salt may be a product stream for external sale or a recycle stream for internal reuse in other instances, or a feed stream for downstream process in other examples. The elemental bromine may be dried in a manner that will be evident to those of ordinary skill in the art prior to its sale or reuse. Certain embodiments of the methods of the invention are described below. Although figures are provided that schematically show certain aspects of the processes of the present invention, these figures should not be viewed as limiting on any particular process of the invention.

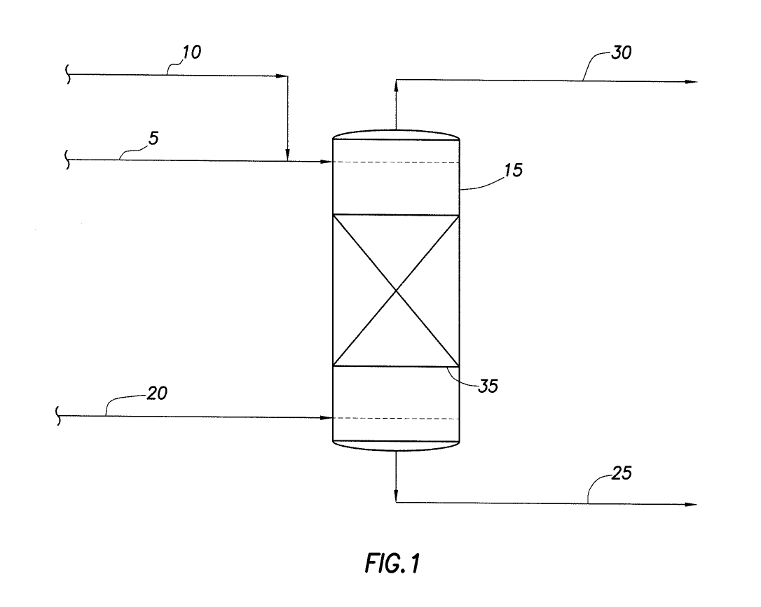

Referring now to FIG. 1, a process for the oxidation of an aqueous metal bromide salt is illustrated in accordance with embodiments of the present invention. In the illustrated embodiment, a stream 5 comprising an aqueous metal bromide salt may be combined with make-up water stream 10 and introduced into a wet oxidation reactor 15 preferably at or near the top thereof. As illustrated, a gaseous oxidant stream 20 may be introduced into the wet oxidation reactor 15 preferably at or near the bottom thereof. In the wet oxidation reactor 15, the metal bromide salt may be oxidized to yield at least elemental bromine and other oxidation products of the metal bromide salt, such as metal hydroxides and metal oxides. A partially oxidized liquid stream 25 comprising the metal hydroxide, metal oxide, or mixtures of one or more of these species may be withdrawn from the wet oxidation reactor 15 preferably at or near the bottom thereof. A gaseous bromine stream 30 comprising the elemental bromine may be withdrawn from the wet oxidation reactor 15 preferably at or near the top thereof.

The stream 5 comprising the aqueous metal bromide salt may be introduced into the wet oxidation reactor 15 preferably at or near the top thereof. The wet oxidation reactor 15 may include a liquid distributor or manifold to more evenly distribute the aqueous metal bromide salt through the internal, cross-sectional area of the wet oxidation reactor 15. The stream 5 may be introduced, for example, at a temperature of about 140° C. to about 190° C. and a pressure of about 3 bars to about 20 bars. In some embodiments, the stream 5 may be introduced at a temperature of about 180° C. to about 190° C. In some embodiments, the stream 5 may be introduced at a pressure of about 7 bars to about 9 bars. In accordance with present embodiments, the stream 5 may be an aqueous stream having a metal bromide salt dissolved therein. For example, the stream 5 may be an aqueous stream having ferrous bromide dissolved therein. In some embodiments, the stream 5 may comprise about 40 weight percent (wt %) to about 60 wt % metal bromide salt. By way of example, the stream 5 may comprise about 40 wt % to about 60 wt % ferrous bromide or, alternatively, comprise about 3 molarity (“M”) to about 6 M ferrous ions. The stream 5 may also contain some oxidized species. For example, the stream 5 may contain ferric species, such as ferric bromide and/or ferric hydroxide. Furthermore, addition of the stream 5 is not limited to an upper section of the wet oxidation reactor 15. For example, the stream 5 may be introduced in a middle section or lower section of the wet oxidation reactor 15 in alternative embodiments with make-up stream 10 introduced into an upper section (above where the stream 5 was introduced) for further washing of the bromine gas leaving the wet oxidation reactor 15, if necessary.

The make-up water stream 10 may be introduced into the wet oxidation reactor 15, preferably at or near the top thereof. The wet oxidation reactor 15 may include a liquid distributor or manifold to more evenly distribute the make-up water stream 10 through the internal, cross-sectional area of the wet oxidation reactor 15. The make-up water stream 10 may be added to maintain, for example, a water-to-Fe molar ratio in the wet oxidation reactor 15 of about 4 to about 10 and, alternatively, of about 4 to about 6. While the make-up water stream 10 and the stream 5 are illustrated as being combined prior to introduction into wet oxidation reactor 15, it should be understood that the present embodiments encompass processes in which these streams are separate feeds to the wet oxidation reactor 15. Furthermore, addition of the make-up water stream 10 is not limited to an upper section of the wet oxidation reactor 15. For example, the make-up water stream 10 may be introduced in a middle section or lower section of the wet oxidation reactor 15 in alternative embodiments.

The gaseous oxidant stream 20 may be introduced into the wet oxidation reactor 15, preferably at or near the bottom thereof. The gaseous oxidant stream 20 may be a stream containing, for example, pure or substantially pure oxygen, air, air to which oxygen has been added, air that contains a reduced concentration of nitrogen, or ozone. The gaseous oxidant stream 20 may be introduced, for example, at a temperature of about 140° C. to about 190° C. and a pressure of about 3 bars to about 20 bars. In some embodiments, the gaseous oxidant stream 20 may be introduced at a temperature of about 180° C. to about 190° C. In some embodiments, the gaseous oxidant stream 20 may be introduced at a pressure of about 7 bars to about 9 bars.

With continued reference to FIG. 1, the wet oxidation reactor 15 contains a packed bed 35 of suitable packing material in accordance with embodiments of the present invention. In the illustrated embodiment, the wet oxidation reactor 15 operates counter currently with the aqueous metal bromide salt, stream 5, being distributed down the packed bed 35 and contacting the upward flowing gaseous oxidant stream 20. While FIG. 1 illustrates only one section of the packed bed 35, embodiments may include more than one section of the packed bed 35 as will be evident to those of ordinary skill in the art. The packed bed 35 may be any of a variety different packing materials including, without limitation, activated carbon, ceramics, polytetrafluoroethylene (“PTFE”), and combinations of these materials. In the case of activated carbon, the activated carbon may be used as a fixed bed or finely ground and dispersed into the metal bromide-rich solution. In some embodiments, the activated carbon may be doped with a suitable metal, such as platinum, palladium, copper, or cobalt, to increase the rate of reaction and further reduce the residence time, thus decreasing reactor size. The metal-doped activated carbon may be arranged in a fixed bed or finely ground and dispersed into solution, for example.

The wet oxidation reactor 15 may generally be operated at a temperature of about 140° C. to about 190° C. and a pressure of about 3 bars to about 20 bars. In some embodiments, the wet oxidation reactor 15 may be operated at a temperature of about 180° C. to about 190° C. In some embodiments, the wet oxidation reactor 15 may be operated at a pressure of about 7 bars to about 9 bars. Residence time of gaseous reactants in the wet oxidation reactor 15 may be generally between about 0.5 minutes to about 10 minutes and, alternatively, about 0.7 minutes to about 2 minutes.

In the wet oxidation reactor 15, the metal bromide salt may be oxidized to yield elemental bromine and metal hydroxide, metal oxide, or mixtures of these species. In the case of the metal bromide salt being ferrous bromide, the oxidation of ferrous ions to ferric ions of mainly ferric hydroxide and ferric bromide is believed to occur in accordance with the following general reaction,

FeBr_{2(aq)}+¼O_{2(aq)}+½H₂O₍ₗ₎→⅔FeBr_{3(aq)}+⅓Fe(OH)_{3(c)} (1)

When excess HBr presents in the liquid feeding reactor 15, the following reaction is also believed to occur,

⅓Fe(OH)_{3(c)}+HBr_{(aq)}→⅓FeBr_{3(aq)}+H₂O₍ₗ₎ (2)

Therefore, when reaction (2) also takes place, the net reaction in the wet air oxidation reactor could be,

FeBr_{2(aq)+}¼O_{2(aq)}+HBr_{(aq)}→FeBr_{3(aq)}+H₂O₍ₗ₎ (3)

In addition to this oxidation reaction, the following thermal decomposition reaction of the ferric bromide is believed to also occur to generate elemental bromine, which flows upward with the oxidant stream 20.

FeBr_{3(aq)}→FeBr_{2(aq)}+½Br_{2(g)} (4)

To ensure an oxidation rate that is relatively high and constant, thus reducing residence time, the wet oxidation reactor 15 may be operated with incomplete oxidation. In some embodiments, about 80% to about 99% of the ferrous ions may be oxidized. In alternative embodiments, the wet oxidation reactor 15 may oxidize from about 80% to about 95% of the ferrous ions resulting in the partially oxidized liquid stream 25 having about 0.5 M to about 1.5 M ferrous ions. In some embodiments, the unconverted ferrous ions in the partially oxidized liquid stream 25 may recycle back to upstream of the HBr neutralizer (not shown) and then be recycled back to the wet oxidation reactor 15.

The above reactions may result in a mildly exothermic heat of reaction. To ensure that the wet oxidation reactor 15 is maintained in the preferred operating temperature range of about 140° C. to about 190° C., the heat generated via reaction may be dissipated by vaporizing water at the top of reactor 15. It is also desirable to restrict the generation of heat in the reactor 15. In this manner, the use of less expensive materials of construction for the wet oxidation reactor 15 may be used while also having a relatively high rate of reaction ensuring the generation of bromine. Restriction of heat generation may also reduce the need for heat exchangers to cool the wet oxidation reactor 15. As illustrated, no heat exchangers are used in cooling the wet oxidation reactor 15.

In some embodiments, dissipation of heat in the wet oxidation reactor 15 may be achieved by evaporating water. The amount of water evaporated and temperature of the exiting gaseous bromine stream 30 depends on a number of factors, including the operating pressure of the wet oxidation reactor 15. A lower operating pressure will evaporate excess water that needs to be replenished. To replenish the evaporated water, embodiments may include the addition of the make-up water stream 10 at the top of the wet oxidation reactor 15 as previously described. The wet oxidation reactor 15 may be operated with a water-to-Fe molar ratio of about 4 to about 10 and, alternatively, of about 4 to about 6, thus preventing the solution reaching its solubility limit and precipitating out of solution.

As previously mentioned, the wet oxidation reactor 15 may be operated at a pressure of about 3 bars to about 20 bars, for example. In addition to the above factors, the operating pressure in the wet oxidation reactor 15 may also be selected to provide a more economically favorable process by balancing a number of factors, including without limitation: (1) increased partial pressure of oxygen which increases the dissolved oxygen content and the rate of oxidation resulting in a smaller reactor size; and (2) higher oxygen pressure which results in higher capital expenditure for the air compressor producing the oxidant stream 20.

The partially oxidized liquid stream 25 may be withdrawn from at or near the bottom of the wet oxidation reactor 15, for example. In some embodiments, the partially oxidized liquid stream 25 may be withdrawn at a temperature of about 130° C. to about 170° C. and, alternatively, about 140° C. to about 160° C. The partially oxidized liquid stream 25 may comprise, for example, the liquid oxidation products from oxidation of the metal bromide salt, including the metal hydroxide, metal oxide, or mixtures of one or more of these species. The partially oxidized liquid stream 25 may also comprise the unconverted metal bromide salt that is not oxidized in the wet oxidation reactor 15. In the case of ferrous bromide, the partially oxidized liquid stream 25 may comprise, for example, aqueous ferric hydroxide and ferric bromide, as well as the unconverted ferrous bromide.

The gaseous bromine stream 30 may be withdrawn from at or near the top of the wet oxidation reactor 15, for example. In some embodiments, the gaseous bromine stream 30 may be withdrawn at a temperature of about 140° C. to about 190° C. and, alternatively, about 160° C. to about 180° C. The gaseous bromine stream 30 may comprise the oxygen, nitrogen, elemental bromine, and/or water vapor. The water vapor, oxygen, and/or nitrogen may be separated out from bromine in a manner evident to those of ordinary skill in the art prior to the sale or reuse of the bromine.

In accordance with embodiments of the present invention, the processes described above with respect to FIG. 1 for oxidation of aqueous metal bromide salts may be used in a bromine-based process for converting lower molecular weight alkanes to higher molecular weight hydrocarbons. For example, HBr produced in the bromine-based process may be neutralized to form the aqueous metal bromide salt, which can then be oxidized as previously described.

The term “higher molecular weight hydrocarbons” as used herein refers to hydrocarbons comprising a greater number of carbon atoms than one or more components of the feedstock. For example, natural gas is typically a mixture of light hydrocarbons, predominately methane, with lesser amounts of ethane, propane, and butane, and even smaller amounts of longer chain hydrocarbons such as pentane, hexane, etc. When natural gas is used as a feedstock, higher molecular weight hydrocarbons produced in accordance with embodiments of the present invention may include a hydrocarbon comprising C2 and longer hydrocarbon chains, such as propane, butane, C5+ hydrocarbons, aromatic hydrocarbons, and mixtures thereof. In some embodiments, part or all of the higher molecular weight hydrocarbons may be used directly as a product (e.g., LPG, motor fuel, etc.). In other instances, part or all of the higher molecular weight hydrocarbons may be used as an intermediate product or as a feedstock for further processing. In yet other instances, part or all of the higher molecular weight hydrocarbons may be further processed, for example, to produce gasoline grade fuels, diesel grade fuels, and fuel additives. In some embodiments, part or all of the higher molecular weight hydrocarbons obtained by the processes of the present invention can be used directly as a motor gasoline fuel having a substantial aromatic content, as a fuel blending stock, or as feedstock for further processing such as an aromatic feed to a process producing aromatic polymers such as polystyrene or related polymers.

The end use of the higher molecular weight hydrocarbons may depend on the particular catalyst employed in the oligomerization portion of the methods discussed below, as well as the operating parameters employed in the process. Other uses should be evident to those skilled in the art with the benefit of this disclosure.

Lower molecular weight alkanes may be used as a feedstock in the processes described herein for the production of higher molecular weight hydrocarbons. A suitable source of lower molecular weight alkanes may be natural gas. As used herein, the term “lower molecular weight alkanes” refers to methane, ethane, propane, butane, pentane or mixtures of two or more of these individual alkanes. The lower molecular weight alkanes may be from any suitable source, for example, any source of gas that provides lower molecular weight alkanes, whether naturally occurring or synthetically produced. Examples of sources of lower molecular weight alkanes for use in the processes of the present invention include, but are not limited to, natural gas, coal-bed methane, regasified liquefied natural gas, gas derived from gas hydrates and/or clathrates, gas derived from anaerobic decomposition of organic matter or biomass, gas derived in the processing of tar sands, and synthetically produced natural gas or alkanes. An example of a suitable source of natural gas includes shale gas, which is natural gas produced from shale formations. Combinations of these may be suitable as well in some embodiments. In some embodiments, it may be desirable to treat the feed gas to remove undesirable compounds, such as sulfur compounds and carbon dioxide.

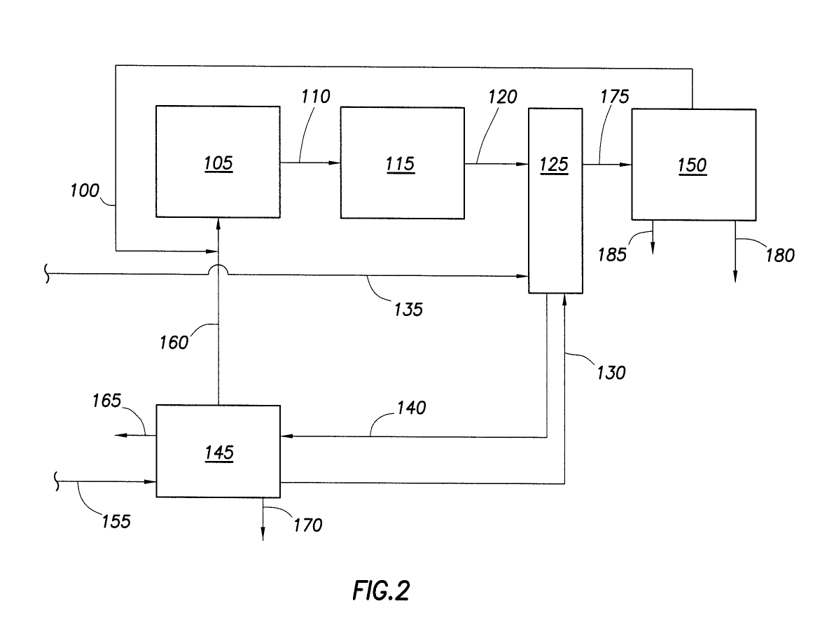

FIG. 2 is a schematic diagram illustrating a process for converting lower molecular weight alkanes to higher molecular weight hydrocarbons in accordance with embodiments of the present invention. In the illustrated embodiment, a gas stream 100 comprising lower molecular weight alkanes comprised of a mixture of feed gas plus a recycled gas stream and a stream 160 of a substantially dry bromine vapor may be reacted in an alkyl bromination stage 105 to produce alkyl bromides and HBr. The resultant alkyl bromides and HBr may then be withdrawn from the bromination stage 105 via line 110 and fed to a synthesis stage 115. In the synthesis stage 115, the alkyl bromides may be reacted over a suitable catalyst in the presence of HBr to produce higher molecular weight hydrocarbons and additional HBr. Those of ordinary skill in the art should appreciate, with the benefit of this disclosure, that the particular higher molecular weight hydrocarbons produced will be dependent, for example, upon the catalyst employed in the synthesis stage 115, the composition of the alkyl bromides introduced into the synthesis stage 115, and the exact operating parameters employed in the synthesis stage 115. Catalysts that may be employed the synthesis reactor used in the synthesis stage 115 include synthetic crystalline alumino-silicate catalyst, such as a zeolite catalyst, as should be recognized by those of ordinary skill in the art with the benefit of this disclosure.

The synthesis effluent stream 120 may be withdrawn from the synthesis stage 115 and fed to the HBr neutralization stage 125. In the HBr neutralization stage 125, the mixture of HBr and higher molecular weight hydrocarbons in the synthesis effluent stream 120 may be contacted with partially oxidized liquid stream 130, which may comprise oxidation products of a metal bromide salt, such as metal hydroxides, metal oxides, or mixtures of one or more of these species, to absorb and neutralize the HBr forming an aqueous liquid comprising a metal bromide salt according to the following reactions:

Fe(OH)_{3(c)}+HBr_{(aq)}→⅓FeBr_{3(aq)}+H₂O₍ₗ₎ (5)

HBr_{(g)}+H₂O₍ₗ₎→HBr_{(aq)} (6)

The resulting aqueous liquid comprising the metal bromide salt can also be contacted with feed gas stream 135 in this HBr neutralization stage 125, for example, to strip out any absorbed residual higher molecular weight hydrocarbons from the aqueous liquid. In some embodiments, the feed gas stream 135 may comprise lower molecular weight alkanes, such as natural gas, for example. In an alternative embodiment, the feed gas stream 135 may be fed into an acid gas (CO₂ and H₂S) removal, dehydration and product recovery unit 150 instead of the HBr neutralization stage 125.

With continued reference to FIG. 2, an aqueous stream 140 comprising the metal bromide salts in an aqueous HBr solution may be removed from the HBr neutralization stage 125 and conveyed to a bromide oxidation stage 145, which comprises a wet oxidation reactor for oxidizing the metal bromide salt (e.g. ferrous bromide), as previously discussed in detail above with respect to FIG. 1 and according to equation 1 to 4 or 1 and 4. In the bromide oxidation stage 145, the metal bromide salt in the aqueous stream 140 may be oxidized with oxidant stream 155 to yield elemental bromine entrained with spent oxidant and moisture. The moisture and spent oxidant may be removed from the elemental bromine in a manner that will be evident to those of ordinary skill in the art with spent oxidant stream 165 and water stream 170 being removed from the bromide oxidation stage 145, for example. The bromide oxidation stage 145 may also yield partially oxidized liquid stream 130 comprising oxidation products of the metal bromide and hydroxide, as well as unconverted metal bromide salt, which may be reused to absorb and neutralize HBr in the HBr neutralization stage.

A hydrocarbon stream 175 comprising the feed gas and higher molecular weight hydrocarbons produced in the synthesis stage 115 may be removed from the HBr neutralization stage 125 and may be conveyed to the dehydration and product recovery unit 150 wherein water may be removed from the remaining constituents. The hydrocarbon stream 175 may also contain residual hydrocarbons that pass through the bromination stage 105 and the synthesis stage 115. In the dehydration and product recovery unit 150, at least a portion of the higher molecular weight hydrocarbons may be recovered as a liquid hydrocarbon product. For example, one or more hydrocarbon product streams 180 comprising higher molecular weight hydrocarbons may be withdrawn from the dehydration and production recovery unit 150 for use as a fuel, a fuel blend, of for further petrochemical processing, for example. The hydrocarbon product streams 180 may also comprise C2+ hydrocarbons from the feed gas stream 135. In addition, lower molecular weight hydrocarbons (e.g., C1-C3 hydrocarbons, such as methane, ethane, and/or propane) may be recovered and recycled to the bromination stage 105 via gas stream 100. Water stream 185 may also be removed from this unit 150. Any suitable method of dehydration and product recovery may be used in the dehydration and product recovery unit 150, including, but not limited to, solid-bed desiccant adsorption followed by refrigerated condensation, cryogenic separation, or circulating absorption oil or some other suitable solvent.

To facilitate a better understanding of the present invention, the following examples of certain aspects of some embodiments are given. The following examples should not be read or construed in any manner to limit, or define, the entire scope of the invention.

### Example 1

Simulations were conducted to analyze the use of a packed bed wet air oxidation reactor for the oxidation of an aqueous stream comprising a ferrous bromide salt. A 2.8 M ferrous bromide solution at 160° C. is fed to the top of a wet air oxidation reactor at a rate of 100 tons per hour (“t/h”) and is distributed over the packing. The ferrous bromide solution has a molar composition as follows: 77.1% H₂O, 15.3% Br—, 4.8% Fe²⁺, and 2.8 FeBr²⁺. Compressed air at 10 barg and 150° C. is fed to the bottom of the wet air oxidation reactor at a rate of 8.6 t/h and is distributed up the packing. The compressed air flowing upwardly through the reactor counter-currently contacts the ferrous bromide solution flowing downward through the reactor over the packed bed. Oxidation of the ferrous ions to ferric ions occurs, producing mainly ferric bromide and ferric hydroxide. At the reactor temperature, the ferric bromide in solution dissociates to ferrous bromide by evolving bromine gas. The overall reaction is exothermic, with the temperature in the reactor being intentionally controlled to dissipate the heat of reaction by vaporizing water.

Effluent gas leaves the top of the reactor at 181° C. and comprises unconverted oxygen, nitrogen, bromine, and water with the following respective molar composition: 1.0% O₂, 44.8% N₂, 9.4% Br₂, and 44.8% H₂O. A partially oxidized liquid stream leaves the bottom of the reactor at 163° C. with the ferrous ions from the ferrous bromide solution being 86% oxidized with the concentration of ferrous ions in the liquid stream reduced to 0.5 M. The water-to-Fe molar ratio in the liquid phase is reduced from 10 in the ferrous bromide solution fed to the reactor to 8.5 in the partially oxidized liquid stream removed from the reactor.

### Example 2

Additional simulations were conducted to analyze the wet air oxidation reactor of Example 1 operating at a lower pressure, which causes increased evaporation of water from the solution. To maintain the water-to-Fe molar ratio of 4 to 10 in the reactor, make-up water is added to the reactor in this example.

A 2.8 M ferrous bromide solution at 160° C. is fed to the top of a wet air oxidation reactor at a rate of 100 t/h and is distributed over the packing. The ferrous bromide solution has a molar composition as follows: 77.1% H₂O, 15.3% Br, 4.8% Fe²⁺, and 2.8 FeBr²⁺. Compressed air at 5 barg and 150° C. is fed to the bottom of the wet air oxidation reactor at a rate of 8.6 t/h and is distributed up the packing. The compressed air flowing upwardly through the reactor counter-currently contacts the ferrous bromide solution flowing downward through the reactor over the packed bed. Oxidation of the ferrous ions to ferric ions occurs, producing mainly ferric bromide and ferric hydroxide. At the reactor temperature, the ferric bromide in solution dissociates to ferrous bromide by evolving bromine gas. The overall reaction is exothermic, with the temperature in the reactor being intentionally controlled to dissipate the heat of reaction by vaporizing water. Make-up water is added to the ferrous bromide solution feeding the top of the reactor at a rate of 6.0 t/h.

Effluent gas leaves the top of the reactor at 159° C. and comprises unconverted oxygen, nitrogen, bromine, and water with the following respective molar composition: 0.9% O₂, 39.4% N₂, 7.2% Br₂, and 52.5% H₂O. A partially oxidized liquid stream leaves the bottom of the reactor at 137° C. with the ferrous ions from the ferrous bromide solution being 95% oxidized with the concentration of ferrous ions in the liquid stream reduced to 0.5 M. The water-to-Fe molar ratio in the liquid phase is reduced from 10 in the ferrous bromide solution fed to the reactor to 9.6 in the partially oxidized liquid stream removed from the reactor. It should be noted that the reactor temperature in Example 2 is lower than Example 1 due to the lower operating pressure.

The results of Examples 1 and 2 are summarized in the table below.

TABLE 1

| | | Example 1 | Example 2 |
|---|---|---|---|
| (1) Feed - Ferrous Solution | | | |
| Flow | t/h | 100 | 100 |
| Temperature | ° C. | 160 | 160 |
| Pressure | barg | 10 | 5 |
| Ion Composition | | | |
| H₂O | mol % | 77.1 | 77.1 |
| Br⁻ | mol % | 15.3 | 15.3 |
| Fe²⁺ | mol % | 4.8 | 4.8 |
| FeBr²⁺ | mol % | 2.8 | 2.8 |
| Ferrous Ion Concentration | mol/dm³ | 2.8 | 2.8 |
| Water-to-Fe ratio | mol/mol | 10 | 10 |
| (2) Feed - Compressed Air | | | |
| Flow | t/h | 8.6 | 8.6 |
| Temperature | ° C. | 150 | 150 |
| Pressure | barg | 10.0 | 5 |
| (3) Feed - Make-Up Water | | | |
| Flow | t/h | 0 | 6 |
| Temperature | ° C. | — | 43 |
| (4) Product - Ferric Solution | | | |
| Flow | t/h | 88.4 | 93.9 |
| Temperature | ° C. | 163 | 137 |
| Pressure | barg | 10 | 5 |
| Ion Composition | | | |
| H₂O | mol % | 77.1 | 78.4 |
| Br⁻ | mol % | 13.8 | 13.3 |
| Fe²⁺ | mol % | 0.8 | 0.3 |
| FeBr²⁺ | mol % | 3.3 | 2.7 |
| Fe(OH)²⁺ | mol % | 2.3 | 3.2 |
| Fe(OH)₂⁺ | mol % | 1.0 | 1.0 |
| Fe(OH)₃ | mol % | 1.7 | 1.1 |
| Ferrous Ion Concentration | mol/dm³ | 0.2 | 0.5 |
| Water-to-Fe ratio | mol/mol | 8.5 | 9.6 |
| (5) Product - Effluent Gas | | | |
| Flow | t/h | 21.0 | 21.5 |
| Temperature | ° C. | 181 | 159 |
| Pressure | barg | 9.3 | 4.3 |
| Ion Composition | | | |
| H₂O | mol % | 44.8 | 52.5 |
| Br₂ | mol % | 9.4 | 7.2 |
| N₂ | mol % | 44.8 | 39.4 |
| O₂ | mol % | 1.0 | 0.9 |
| Reactor Sizing | | | |
| Residence Time | minutes | 1.0 | 1.0 |
| Packing Type | | Pall Ring | Pall Ring |
| | | 1½″ | 1½″ |
| Diameter | m | 1.6 | 2.0 |
| Packing Height | m | 14.3 | 17.5 |

Certain embodiments of the methods of the invention are described herein. Although major aspects of what is to believed to be the primary chemical reactions involved in the methods are discussed in detail as it is believed that they occur, it should be understood that side reactions may take place. One should not assume that the failure to discuss any particular side reaction herein means that that reaction does not occur. Conversely, those that are discussed should not be considered exhaustive or limiting. Additionally, although figures are provided that schematically show certain aspects of the methods of the present invention, these figures should not be viewed as limiting on any particular method of the invention.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Although individual embodiments are discussed, the invention covers all combinations of all those embodiments. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. All numbers and ranges disclosed above may vary by some amount. Whenever a numerical range with a lower limit and an upper limit is disclosed, any number and any included range falling within the range are specifically disclosed.

What is claimed is:

1. A process comprising:
oxidizing a stream comprising a dissolved metal bromide salt in a wet oxidation reactor comprising a packed section to produce at least a partially oxidized liquid stream comprising oxidized products of the metal bromide salt and a gaseous bromine stream comprising elemental bromine.

2. The process of claim 1 further comprising adding a make-up water stream to the wet oxidation reactor to maintain a water-to-Fe molar ratio of about 4 to about 10 in the partially oxidized liquid stream.

3. The process of claim 2 wherein the water-to-Fe molar ratio is maintained at about 4 to about 6.

4. The process of claim 2 wherein the make-up water stream is combined with the stream prior to addition to the wet oxidation reactor.

5. The process of claim 1 wherein the metal bromide salt comprises a metal selected from the group consisting of Fe(II), Cu(I), and mixtures thereof.

6. The process of claim 1 wherein the oxidized products of the metal bromide salt comprise at least one product selected from the group consisting of a metal hydroxide, a metal oxide, and a combination thereof.

7. The process of claim 1 wherein the wet oxidation reactor is operated at a temperature of about 140° C. to about 190° C. and a pressure of about 3 bars to about 20 bars.

8. The process of claim 1 wherein the dissolved metal bromide salt comprises ferrous bromide, the stream comprising about 40 weight percent to about 60 weight percent ferrous bromide.

9. The process of claim 8 wherein about 80% to about 99% of ferrous ions in the stream are oxidized.

10. The process of claim 1 further comprising introducing a gaseous oxidant stream to the wet oxidation reactor, the gaseous oxidant stream comprising at least one oxidant selected from the group consisting of oxygen, ozone, and combinations thereof.

11. The process of claim 1 wherein the packed section comprises at least one packing material selected from the group consisting of activated carbon, doped activated carbon, a ceramic, polytetrafluoroethylene, and combinations thereof.

12. The process of claim 1 further comprising:
reacting lower molecular weight alkanes and bromine to produce at least alkyl bromides and hydrogen bromide;
reacting the alkyl bromides over a catalyst to produce at least higher molecular weight hydrocarbons and additional hydrogen bromide; and
neutralizing the hydrogen bromide and the additional hydrogen bromide to produce at least the stream that is oxidized.

13. The process of claim 12 wherein the lower molecular weight alkanes comprise methane, and wherein the higher molecular weight alkanes comprise hydrocarbons having four or more carbon atoms.

14. A process comprising:
reacting lower molecular weight alkanes and bromine to produce at least alkyl bromides and hydrogen bromide, wherein the lower molecular weight alkanes comprise methane;
reacting the alkyl bromides over a catalyst to produce at least higher molecular weight hydrocarbons and additional hydrogen bromide, wherein the higher molecular weight hydrocarbons comprise hydrocarbons having four or more carbon atoms;
neutralizing the hydrogen bromide and the additional hydrogen bromide in an aqueous stream of ferric hydroxide and ferrous bromide to produce at least a ferrous/ferric stream comprising a dissolved ferrous bromide and dissolved ferric bromide in a concentration of about 40 weight percent to about 60 weight percent; and
oxidizing the ferric/ferrous stream in a wet oxidation reactor comprising a packed section to produce at least a partially oxidized liquid stream comprising ferric bromide, wherein the wet oxidation reactor operates at a temperature of about 140° C. to about 190° C. and a pressure of about 3 bars to about 20 bars.

15. The process of claim 14 further comprising adding a make-up water stream to the wet oxidation reactor to maintain a water-to-Fe molar ratio of about 4 to about 10 in the partially oxidized liquid stream, and wherein heat exchangers are not used to cool the wet oxidation reactor.

16. The process of claim 15, wherein the water-to-Fe molar ratio is maintained at about 4 to about 6.

17. The process of claim 15 wherein the make-up water stream is combined with the ferrous/ferric stream prior to addition to the wet oxidation reactor.

18. The process of claim 14, wherein about 80% to about 99% of ferrous ions in the ferrous/ferric stream are oxidized.

19. The process of claim 14 wherein the wet oxidation reactor is operated at a temperature of about 180° C. to about 190° C. and a pressure of about 7 bars to about 9 bars.

20. A system comprising:
a wet oxidation reactor having an inlet in an upper end thereof for an aqueous metal bromide salt, an outlet in the lower end thereof for an oxidant stream, and one or more packed sections, wherein the wet oxidation reactor is configured to countercurrently contact the aqueous metal bromide salt with the oxidant stream over the one or more packed sections so as to oxidize the aqueous metal bromide salt and produce at least a partially oxidized liquid stream comprising oxidized products of the aqueous metal bromide salt and a gaseous bromine stream comprising elemental bromine.
